(19) United States
(12) Patent Application Publication
(10) Pub. No.: US 2010/0329977 A1
(43) Pub. Date: Dec. 30, 2010

(54) METHOD FOR IMAGING TUMOR TISSUE

(76) Inventors: Arne Hengerer, Mohrendorf (DE); Andreas Kappel, Koenigstein (DE)

Correspondence Address: HARNESS, DICKEY & PIERCE, P.L.C., P.O.BOX 8910, RESTON, VA, 20195, US

(21) Appl. No.: 12/820,205

(22) Filed: Jun. 22, 2010

(30) Foreign Application Priority Data
Jun. 24, 2009 (DE) 10 2009 030 321.9

(51) Int. Cl. A61K 51/10; A61K 51/08; A61K 49/00; A61K 49/14; G01N 33/53; G01N 33/573; G01N 33/58; C07K 14/435

(52) U.S. Cl. 424/1.49; 424/1.69; 424/9.1; 424/9.34; 435/7.1; 435/7.4; 435/7.9; 530/350

(57) ABSTRACT

A method is disclosed for imaging a tumor tissue, wherein in at least one embodiment a) the tumor tissue is contacted with a monoclonal antibody, an antigen-binding fragment thereof, a recombinant binding protein, an aptamer, or other molecule, each of which recognizes and binds at least one neoepitope which has been generated by proteolytic cleavage of proteins surrounding the tumor tissue by tumor-specific proteases, and b) the complexes formed from neoepitope and monoclonal antibody, antigen-binding fragment thereof, recombinant binding protein, aptamer, or other molecule are depicted with an imaging method, and also to neoepitopes generated by proteolytic cleavage of surrounding proteins by tumor-specific proteases, and also proteins or peptides for generating neoepitopes by tumor-specific proteases.

### PRIORITY STATEMENT

The present application hereby claims priority under 35 U.S.C. §119 on German patent application number DE 10 2009 030 321.9 filed Jun. 24, 2009, the entire contents of which are hereby incorporated herein by reference.

### FIELD

At least one embodiment of the invention generally relates to a method for imaging a tumor tissue. At least one embodiment of the invention further generally relates to neoepitopes which have been generated by proteolytic cleavage of proteins surrounding a tumor tissue by tumor-specific proteases. At least one embodiment of the invention further generally relates to proteins and peptides for generating neoepitopes by tumor-specific proteases.

### BACKGROUND

Cancers which reach a metastatic stage normally have a poor prognosis and are often life-threatening. The pathological metabolic pathway which leads to metastasis formation is characterized by two critical steps: penetration of the basal membrane, through which the metastasizing cells from a primary tumor can migrate into further tissues, and also the onset of tumor angiogenesis (cf., for example, Hanahan, D. and Weinberg, R. A.: The hallmarks of cancer. Cell 100, 57-70 (2000)). Both steps are based on a dysregulation of the complex interaction between the cellular components of a tissue and the surrounding matrix. It is now recognized that proteases, such as the matrix metalloproteinases (MMPs) for example, which in particular are secreted by metastasizing tumor cells, cause the proteolysis of polymeric and nonfibrillar matrix components, which is crucial for the process of metastasis (cf., for example, McCawly, L. J., Matrisian, L. M.: Matrix metalloproteases: They're not just for matrix anymore. Curr. Opin. Cell Biol 13, 534-540 (2001)).

The pathological function and the specific expression of several tumor-derived proteases are well known. These proteases are therefore an attractive potential target (“drug target”) for new chemotherapeutic drugs. The tumor proteases are also attractive targets for the in vivo detection and the molecular imaging of primary and metastasizing tumors (cf., for example, McEntyre, J. O. and Matrisian, L. M.: Molecular imaging of proteolytic activity in cancer. Journal of Cellular Biochemistry 90, pp. 1087-1097 (2003)).

In order to detect the activity of tumor proteases in vivo by imaging techniques, novel contrast agents, so-called smart contrast agents, which are conjugates of a peptide and a fluorophore, were developed (cf., for example, Kumar, S. and Richards-Kortum, R.: Optical molecular imaging agents for cancer diagnostics and therapeutics. Nanomedicine 1, 23-30 (2006)). These agents are substrates for tumor proteases and are converted by proteolytic cleavage from the inactive state (“quenched state”) into the fluorescent state (cf., for example, Weisleder, R. and Ntziachristos, V.: Shedding light onto live molecular targets. Nature Med. 9, 123-128 (2003)). The advantages of these methods are the good accessibility of each substrate for the proteases, the tumor specificity of the expression of the proteases, and the fact that the signal can be intensified over time by cleaving several substrate molecules by a single protease molecule. A major problem of these methods, however, is that they are based on fluorescence detection and are therefore not compatible with imaging techniques such as MRI, PET, and SPECT for example, which are very sensitive and frequently used in clinical practice. Furthermore, these methods have not acquired any significance in clinical practice, probably because of the above-mentioned problems. Other methods are based on using radioactively labeled inhibitors of tumor-specific metalloproteinases. Although the binding of these inhibitors can be tracked with imaging methods, such as PET, used in hospitals, the measured signals are low, since a single enzyme molecule can bind only a single inhibitor molecule (cf., for example, W P LI and C J Anderson: Imaging Matrix Metalloproteinase expression in tumors. Q J Nucl Med 47, 201-208 (2003)).

Furthermore, the use of monoclonal antibodies (mAbs) as the basis for highly specific reagents in depicting a tumor is generally accepted (cf., for example, Stipsanelli, E. and Valsamaki, P.: Old and new trends in breast cancer imaging and therapeutic approach. Hell. J. Nucl. med. 8, 103-108 (2005)). With these methods, mAbs which are specific for target molecules derived from a tumor are customarily conjugated with radionuclides. After administration to the patient, the radionuclide-mAb conjugate accumulates in potential tumor tissues and can thus be visualized, for example by PET or SPECT. A major problem with this method is the low concentration of most tumor antigens, which makes detection often very difficult. Furthermore, most tumor-specific antigens are intracellular proteins, which are difficult to access by the radionuclide-mAb conjugate. An exception here is the human antibody COU-1, which binds to processed intracellular cytokeratin 8/18 (cf., for example, Ditzel H. J. et al.: Cancer-associated cleavage of cytokeratin 8/18 heterotypic complexes exposes a neoepitope in human adenocarcinomas. J. Biol. Chem. 277, 21712-21722 (2002)). This antibody has been successfully used for immunoscintigraphy of various adenocarcinomas, such as colon cancer. The protease responsible for processing intracellular cytokeratins is unknown, but it is believed that this protease is only active in apoptotic cancer cells. The expression of cytokeratin 8/18, however, is limited to epithelial cells, and COU-1 can therefore only be used to detect an adenocarcinoma. In addition to monoclonal antibodies, which are used nowadays mainly as humanized variants, other molecules, such as binding proteins or aptamers for example, which specifically bind to tumor antigens are also suitable for this method.

There is therefore a need for a reliable and sensitive method for depicting a metastasizing tumor tissue. In particular, the method should be easy to carry out and suitable for routine use in general medical practice. The method should further be suitable for depicting a very broad range of tumor types. Furthermore, the method should provide evidence of the metastatic potential or of the spread of the metastasization of a tumor. Also, the method should allow reliable diagnosis regarding the type, stage, and metastatic potential and spread of a cancer with one examination.

It was found that, surprisingly, a combination of tumor diagnostics, in which one or more neoepitopes generated by proteolytic cleavage of proteins surrounding the tumor tissue by tumor-specific proteases are recognized and bound by molecules which specifically bind to the neoepitopes, and an imaging method makes possible a highly specific, sensitive, and information depiction of a tumor tissue, in particular of a metastasizing tumor tissue.

### SUMMARY

In at least one embodiment, the invention accordingly provides a method for imaging a tumor tissue, wherein a) a tumor tissue is contacted with a monoclonal antibody, an antigen-binding fragment thereof, a recombinant binding protein, an aptamer, or other molecule, each of which recognizes and binds at least one neoepitope which has been generated by proteolytic cleavage of proteins surrounding a tumor tissue by tumor-specific proteases, and b) the complexes formed from neoepitope and monoclonal antibody, antigen-binding fragment, recombinant binding protein, aptamer, or other molecule are depicted with an imaging method.

At least one embodiment of the invention further provides a method for imaging a tumor tissue, wherein a tumor tissue contacted with a monoclonal antibody, an antigen-binding fragment thereof, a recombinant binding protein, an aptamer, or other binding molecule, each of which recognizes and binds at least one neoepitope which has been generated by proteolytic cleavage of proteins surrounding the tumor tissue by tumor-specific proteases, is subjected to an imaging method. Here, the tumor tissue can be present in vivo or in vitro. Prior to this method, the step of contacting the tumor tissue with a monoclonal antibody, an antigen-binding fragment thereof, a recombinant binding protein, an aptamer, or other molecule, each of which specifically binds to the neoepitope to be detected, can take place in vivo or in vitro according to methods known per se.

At least one embodiment of the invention further provides a method for imaging a tumor tissue, wherein a complex of at least one neoepitope generated by proteolytic cleavage of proteins surrounding a tumor tissue by tumor-specific proteases, and at least one monoclonal antibody, antigen-binding fragment thereof, recombinant binding protein, aptamer, or other binding molecule, each of which is specific for the neoepitope, is depicted with an imaging method. The complex of at least one neoepitope and one monoclonal antibody, antigen-binding fragment thereof, recombinant binding protein, aptamer, or other binding molecule, each of which is specific to the neoepitope generated by proteolytic cleavage of proteins surrounding a tumor tissue by tumor-specific protease, may be present in vivo or in vitro in a tissue or a tissue sample and is prepared prior to carrying out the method according to methods known per se.

At least one embodiment of the invention further provides for the use of a monoclonal antibody, an antigen-binding fragment thereof, a recombinant binding protein, an aptamer, or other binding molecule, each of which recognizes and binds at least one neoepitope which has been generated by proteolytic cleavage of proteins surrounding a tumor tissue by tumor-specific proteases, together with an imaging method for imaging a tumor tissue. The invention further provides for the use of neoepitopes generated by proteolytic cleavage of proteins surrounding a tumor tissue by tumor-specific proteases and having the formulae below together with a monoclonal antibody, an antigen-binding fragment thereof, a recombinant binding protein, an aptamer, or other binding molecule, each of which recognizes and binds at least one of the neoepitopes below, together with an imaging method for imaging a metastasizing tumor tissue.

At least one embodiment of the invention further provides for the use of the proteins or peptides mentioned below for generating neoepitopes by tumor-specific proteases together with a monoclonal antibody, an antigen-binding fragment thereof, a recombinant binding protein, an aptamer, or other binding molecule, each of which recognizes at least one neoepitope generated from the above proteins or peptides by proteolytic cleavage by tumor-specific proteases, together with an imaging method for imaging a metastasizing tumor tissue.

At least one embodiment of the invention further provides neoepitopes generated by proteolytic cleavage of proteins surrounding a tumor tissue by tumor-specific proteases, for example, the epitopes generated by cleavage of MMP-7 and having the C-terminal sequence Lys-Pro-Leu-Glu, or the epitopes generated by cleavage of MMP-7 and having the C-terminal sequence Lys-Leu-Pro-Ala.

At least one embodiment of the invention further provides an extracellular protein or peptide for generating neoepitopes by tumor-specific proteases, for example, neoepitopes of the following proteins or peptide sequences derived from them: collagen I-IV, collagen VI-X, fibronectin, laminin, elastin, proteoglycan core protein, pro-MMP2, pro-MMP9, aggrecan, gelatin, and similar substrate molecules, and also synthetic substrate molecules optimized with regard to the recognition of the neoepitopes resulting from their cleavage by cognate binders, half life in the body, bioavailability, and pharmacokinetics and pharmacodynamics.

At least one embodiment of the invention further provides for detecting tumor-specific proteases, for example, MMP-1, MMP-2, MMP-3, MMP-7, MMP-10, MMP-11, MMP-12, MMP-14, MMP-15, MMP-16, MMP-17, uPA, tPA, and further proteases.

At least one embodiment of the method described above for imaging a tumor tissue also allows the detection of proteases involved in the proteolytic cleavage of proteins surrounding a tumor tissue. The formation of a complex of neoepitope and monoclonal antibody, antigen-binding fragment thereof, recombinant binding protein, aptamer, or other molecule, each of which specifically binds to a particular neoepitope, provides evidence of the presence of a specific protease; resulting from this is an indication of the nature of the tumor.

At least one embodiment of the present invention is based on the fact that secreted proteases derived from a tumor specifically cleave endogenous matrix substrates or exogenous peptide or protein substrates administered, for example, intravenously, orally, or by inhalation. The resulting cleaved peptides or proteins have a new N-terminal end and a new C-terminal end, which represent the so-called neoepitopes. Such neoepitopes occur in healthy tissue not at all or are at a lower concentration than in the tumor tissue and are therefore characteristic of tissue which surrounds a tumor and, in particular, a metastasizing tumor. Here, the term “tumor” comprises both benign tumors and malignant tumors. In particular, the term “tumor” comprises cancers and, in particular, metastasizing cancers and carcinomas.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example and will herein be described in detail. It should be understood, however, that there is no intent to limit example embodiments of the present invention to the particular forms disclosed. On the contrary, example embodiments are to cover all modifications, equivalents, and alternatives falling within the scope of the invention.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The term “tumor tissue” means both tissue known to contain a tumor and tissue believed to contain a tumor. The tumor tissue is preferably present in a human, but may also be present in an animal or plant.

The neoepitopes formed as described above can be recognized by a previously administered or added specific monoclonal antibody, antigen-binding fragment thereof, a specific recombinant binding protein, an aptamer, or other binding molecule. These molecules then aggregate in the surroundings of a corresponding tumor and form complexes with the neoepitopes present. Such molecules are administered to the patient to be examined prior to carrying out the method according to the invention depending on the nature of the suspected tumor and the type of neoepitope-binding molecule. This administration takes place in a manner known per se, for example, intravenously, orally, or by inhalation. An intravenous or oral administration is preferred.

Monoclonal antibodies which specifically bind to neoepitopes generated by proteases are well known in the art (for example, in Hughes C. E. et al.: Monoclonal antibodies recognizing protease-generated neoepitopes from cartilage proteoglycan degradation. Application to studies of human link protein cleavage by stromelysin, J. Biol. Chem., 267, 23, 16011-16014 (1992), the entire contents of which are hereby incorporated herein by reference) and are currently used in commercially available diagnostic tests (for example, the Enygnost F1+2mono Test from Siemens). Likewise, antigen-binding fragments for a defined epitope or recombinant binding proteins, aptamers, or other specific binding molecules recognizing a defined epitope are well known in the art and may be prepared according to methods known per se.

The monoclonal antibody, the antigen-binding fragment thereof, a recombinant binding protein, aptamers, or other specific binding molecules recognize at least one neoepitope generated by proteolytic cleavage of proteins surrounding the tumor tissue by tumor-specific proteases. Monospecific antibodies specifically bind to the epitope against which they are directed. Since a tumor protease forms a multiplicity of neoepitopes by repeated cleavage, and consequently a multiplicity of neoepitope-antibody complexes are formed, a signal amplification step arises as a result. Instead of a monoclonal antibody, a suitable antibody fragment which binds the antigen on the neoepitope or a specific recombinant binding protein for the neoepitope or an aptamer or other molecule for the neoepitope which specifically binds to the neoepitope to be detected may also be used.

For detection with an imaging method, the above-mentioned molecules are appropriately coupled to a detectable label. Examples of a detectable label are a fluorophore (FITC, GFP), a radioactive isotope (F18, I-124, C-11), a lanthanide chelate (for example, gadolinium GTPA), or an iron oxide nanoparticle (USPIO, MION, SPIO).

The abovementioned molecules which bind to a neoepitope may also recognize two or more neoepitopes, i.e., the molecules may also be bispecific and bivalent. Examples of such molecules are bivalent monospecific and bispecific diabodies or fragments thereof and also triabodies or tetrabodies. Diabodies comprise variable domains of a heavy chain of a monoclonal antibody connected to the variable domain of a light chain of an antibody on the same polypeptide chain and are thus linked by a peptide linker which is too short to allow pairing between both domains on the same chain. This results in pairing with the complementary domains of another chain, which thus generates the formation of dimeric molecules having two functional antigen-binding sites. The diabodies may also be single-chained. Using bispecific and bivalent antibodies leads to associations of aggregates between these molecules and the neoepitopes. These aggregates may be detected as such without specific labeling with an imaging method. It is, however, also possible to couple these molecules to a detectable label.

Instead of complete antibody molecules, antigen-binding fragments, for example Fab, F(ab′)₂, or Fᵥ, may also be used.

The recombinant binding protein may be any binding protein which recognizes a neoepitope. Preferably, the recombinant binding protein is an sFᵥ molecule, a humanized antibody, or a recombinant protein which specifically binds to the neoepitope. Furthermore, aptamers or other molecules which specifically bind to the neoepitope may be used. Such molecules are known to a person skilled in the art and may be prepared in a manner known per se and using knowledge of the particular epitope. A suitable combination of these molecules may also be used.

Neoepitopes to which the molecules used according to the invention bind are well known in the art. They are, however, only poorly characterized with regard to their sequence and are mostly defined in terms of the antibodies that bind to them (e.g., C E Hughes et al. Monoclonal antibodies that specifically recognize neoepitope sequences generated by ‘aggrecanase’ and matrix metalloproteinase cleavage of aggrecan: application to catabolism in situ and in vitro. Biochem J. 1995; 305: 799-804, the entire contents of which arte hereby incorporated herein by reference).

Furthermore, the following neoepitopes are recognized advantageously according to the invention: neoepitopes originating from collagen I-IV, collagen VI-X, fibronectin, laminin, elastin, proteoglycan core protein, pro-MMP2, pro-MMP9, aggrecan, gelatin, and further substrate molecules.

The neoepitopes targeted and bound according to at least one embodiment of the invention are formed from specific proteases secreted by a tumor tissue by cleavage of endogenous matrix components. Examples of corresponding proteases are MMP-1, MMP-2, MMP-3, MMP-7, MMP-10, MMP-11, MMP-12, MMP-14, MMP-15, MMP-16, MMP-17, uPA, tPA, and further proteases which are known in the art.

The neoepitopes may also be further formed from proteins or peptides or other substrates of the enzymes to be detected which are specifically administered to a patient or tissue to be examined. These proteins or peptides may be optimized with regard to cleavability by the tumor protease to be detected or with regard to recognition of the neoepitopes resulting from their cleavage by cognate binders, but also with regard to other criteria such as, for example, half life in the body, bioavailability, pharmacokinetics and pharmacodynamics, or others. The advantages of using such exogenously added substrates for detecting tumor proteases over endogenous substrates reside in the improved detectability of cleavage by selecting or optimizing the substrate substance according to the abovementioned criteria.

The antibodies, antigen-binding fragments, recombinant binding proteins, aptamers, or other specific binding molecules, accumulated in the tumor tissue owing to binding to the neoepitopes generated by tumor-specific proteases, and the complexes resulting from them can be depicted by an imaging method. Examples of such methods are MRI, PET, SPECT, PET-CT, MR, PET-MR, US, or IR methods. Such methods are well known to a person skilled in the art. Corresponding equipment are commercially available.

A person skilled in the art also has the ability to select the particular method in combination with a suitably labeled antibody, antigen-binding fragment, recombinant binding protein, aptamer, or other molecule which specifically binds to the neoepitope to be detected.

For instance, radionuclide conjugates of monoclonal antibodies recognizing a neoepitope may be detected by means of PET or SPECT, whereas unconjugated bispecific and bivalent monoclonal antibodies which connect neighboring neoepitopes to each other form larger aggregates and can be detected directly. The T2 value of the aggregate changes, compared with the T2 value of the separate epitope, and may thus be easily measured by means of MRI.

Tumor cells may be detected very efficiently by a combination of such imaging techniques with diagnostic molecules which specifically bind neoepitopes formed by tumor proteases. Based on the size of the signal, evidence may also be derived about the aggressiveness, i.e., the metastatic potential or the progression of the metastasization.

The method according to at least one embodiment of the invention is especially suitable for imaging solid tumor types such as, for example, sarcomas, carcinomas, blastomas, malignant melanoma, and others.

The images of tumor tissue obtained with the method according to at least one embodiment of the invention may be used for the diagnosis and therapy of the particular tumor. The image information thus obtained may also find use in connection with surgical methods and/or the targeted therapy of a cancer condition, for example, as a real-time imaging method during a surgical intervention. This information may also be further used for monitoring the progression of the disease or the success of a therapy carried out. The image information may thereby be evaluated both by a trained physician and by a computer.

The method according to at least one embodiment of the invention has the advantages of the smart contrast agents known in the prior art concerning properties optimized for a specific use with regard to cleavage by the tumor protease to be detected, half life in the body, bioavailability, pharmacokinetics and pharmacodynamics, and also concerning access of proteases to the substrate and signal intensification by cleavage of several substrate molecules by a single protease molecule, but, unlike such methods, is compatible with imaging techniques which are frequently used in clinical practice, such as PET, SPECT, and MRI. In addition to signal amplification by protease activity, the major problem of traditional imaging methods using monoclonal antibodies is overcome, namely the low antigen concentration. Since the neoepitopes generated by tumor proteases are present in the extracellular space, these neoepitopes are easily accessible for the antibodies or antigen-binding fragments or recombinant binding proteins used, which, in contrast to diagnostics which are based on other tumor antigens which are located on or in the cell, is a further advantage of the method according to at least one embodiment of the invention.

Since, according to at least one embodiment of the invention, extracellular substrates may also be administered, the effect of the complete spectrum of secreted tumor proteases may be detected, even of those proteases for which no suitable substrate is present in the surrounding tissue. As a result, a broader neoepitope target spectrum may be considered—which enables the identification of stronger neoepitopes, and so there is no restriction to neoepitopes generated from substrates which are present endogenously. Thus, the diagnostic potential of the method according to the invention is not limited to tumor types for which neoepitopes are already known (for example, the abovementioned COU-1 epitope in connection with detection of adenocarcinomas). Thus, any solid tumor tissue is imageable according to the invention and its metastatic potential or extent may be assessed. The method according to the invention thus allows reliable diagnosis or prognosis of a metastasizing tumor or cancer tissue.

At least one embodiment of the invention is more particularly elucidated with the aid of the example of a breast carcinoma. Strong expression of the metalloproteinase MMP-2 by breast carcinomas is associated with a poor prognosis (e.g., Talvensaari-Mattila A., Matrix metalloproteinase-2 (MMP-2) is associated with survival in breast carcinoma. Br J Cancer 89, 1270-1275, 2003, the entire contents of which are hereby incorporated herein by reference). It is important to know for the therapy following initial diagnosis whether and to what extent a breast carcinoma expresses MMP-2 in order to adapt the therapy accordingly, i.e., for example, to introduce a higher-dosed chemotherapy or combined chemotherapy from the beginning. Furthermore, it is important to check whether MMP-2-secreting metastases are already present and to localize these metastases exactly. For this purpose, a patient is, for example, subjected to intravenous administration of a humanized monoclonal antibody which is conjugated with a radionuclide and specifically binds to neoepitopes of matrix proteins cleaved by MMP-2.

Alternatively, a synthetic peptide which is specifically cleaved by MMP-2 may also be injected into the patient some time prior to administering the antibody. In both cases, the substrates supplied extrinsically or the matrix proteins present intrinsically are cleaved by MMP-2 secreted by tumor cells, forming neoepitopes over the uncleaved molecules. In a further step, a binding molecule which is conjugated with a radioactive nuclide and specifically binds to the neoepitopes formed is now applied to the patient. This molecule may be subsequently detected by PET or another imaging method. As a result, the distribution in the body of the labeled binding molecules bound to the neoepitopes and hence, indirectly, of the MMP-2-secreting tumor cells may be depicted visually. As a result, it can be detected whether the primary tumor produces MMP-2 (this is not possible by means of a laboratory test), and metastases possibly present may be localized. There may even be constellations in which the primary tumor secretes only a little MMP-2 or none at all, but metastases starting from the tumor produce MMP-2.

In addition to the initial diagnosis mentioned at the beginning with therapeutic relevance (chemotherapeutic and immunotherapeutic) and diagnostic classification of the disease, the method of an embodiment also provides localization of primary tumors and metastases in patients for surgical and/or nuclear medical methods. Furthermore, repeated use of the method over time provides evidence of the change in the extent of the primary tumor or of metastases during surgical, chemotherapeutic or immunotherapeutic, or nuclear medical therapy, and therefore allows an assessment of the success of the therapy with corresponding consequences.

The patent claims filed with the application are formulation proposals without prejudice for obtaining more extensive patent protection. The applicant reserves the right to claim even further combinations of features previously disclosed only in the description.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

1. A method for imaging a tumor tissue, the method comprising:
a) contacting a tumor tissue with a monoclonal antibody, an antigen-binding fragment thereof, a recombinant binding protein, an aptamer, or other molecule, each of which recognizes and binds at least one neoepitope which has been generated by proteolytic cleavage of proteins surrounding a tumor tissue by tumor-specific proteases; and
b) depicting, with an imaging method, the complexes formed from neoepitope and monoclonal antibody, antigen-binding fragment thereof, recombinant binding protein, aptamer, or other molecule.

2. The method as claimed in claim 1, wherein the proteins surrounding the tumor tissue are endogenous matrix components.

3. The method as claimed in claim 1, wherein the proteins surrounding the tumor tissue are externally administered peptide or protein substrates.

4. The method as claimed in claim 1, wherein the antibody is at least one of a bivalent and bispecific antibody (diabody).

5. The method as claimed in claim 1, wherein the antigen-binding fragment is Fab, F(ab′)₂, or Fᵥ.

6. The method as claimed in claim 1, wherein the recombinant binding protein is an sFᵥ, a humanized antibody, or a recombinant protein comprising a variable region of an antibody.

7. The method as claimed in claim 1, wherein the monoclonal antibody, the antigen-binding fragment thereof, the recombinant binding protein, the aptamer, or other molecule, each of which specifically binds to the neoepitope to be detected, recognizes neoepitopes which are formed by proteases secreted by tumors or metastases and which are from the group of the following matrix proteins: collagen I-IV, collagen VI-X, fibronectin, laminin, elastin, proteoglycan core protein, pro-MMP2, pro-MMP9, aggrecan.

8. The method as claimed in claim 1, wherein the monoclonal antibody, the antigen-binding fragment thereof, the recombinant binding protein, the aptamer, or other molecule, each of which specifically binds to the neoepitope to be detected, is coupled to a detectable label.

9. The method as claimed in claim 8, wherein the detectable label is a fluorophore, a radioactive isotope, an enzyme, a lanthanide chelate, or a chromophore.

10. The method as claimed in claim 1, wherein the imaging method is selected from the group consisting of MRI, PET, SPECT, PET-CT, MR, PET-MR, US and IR.

11. The method as claimed in claim 1, wherein the tumor tissue is a solid tumor, in particular a sarcoma, a carcinoma, a blastoma, or a malignant melanoma.

12. The method as claimed in claim 1, wherein the tumor tissue is a metastasizing tumor tissue.

13. Neoepitopes which are generated by proteolytic cleavage of proteins surrounding a tumor tissue by tumor-specific proteases and which are in matrix proteins from the following group: collagen I-IV, collagen VI-X, fibronectin, laminin, elastin, proteoglycan core protein, pro-MMP2, pro-MMP9, aggrecan.

14. Proteins, peptides, or other molecules, optimized with regard to recognition of neoepitopes resulting from their cleavage by cognate binders, half life in the body, bioavailability, and pharmacokinetics and pharmacodynamics, for generating neoepitopes of the following tumor-specific proteases: MMP-1, MMP-2, MMP-3, MMP-7, MMP-10, MMP-11, MMP-12, MMP-14, MMP-15, MMP-16, MMP-17, uPA, tPA.

15. Use of a method as claimed in claim 1 for detecting a tumor-specific protease.

16. The method as claimed in claim 2, wherein the proteins surrounding the tumor tissue are externally administered peptide or protein substrates.

17. The method as claimed in claim 11, wherein the tumor tissue is a sarcoma, a carcinoma, a blastoma, or a malignant melanoma.

18. Proteins, peptides, or other molecules, optimized with regard to recognition of the neoepitopes of claim 13 resulting from their cleavage by cognate binders, half life in the body, bioavailability, and pharmacokinetics and pharmacodynamics, for generating neoepitopes of the following tumor-specific proteases: MMP-1, MMP-2, MMP-3, MMP-7, MMP-10, MMP-11, MMP-12, MMP-14, MMP-15, MMP-16, MMP-17, uPA, tPA.

19. A method comprising:
using the method as claimed in claim 1 for detecting a tumor-specific protease.
